(19) United States
(12) Patent Application Publication
(10) Pub. No.: US 2020/0398073 A1
(43) Pub. Date: Dec. 24, 2020

(54) SYSTEM AND METHOD FOR AUGMENTING THERAPEUTIC TREATMENT USING LIGHT AND PULSE MODULATION

(71) Applicant: Michael L. McIntyre, Dana Point, CA (US)

(72) Inventor: Michael L. McIntyre, Dana Point, CA (US)

(21) Appl. No.: 17/008,981

(22) Filed: Sep. 1, 2020

Related U.S. Application Data
(63) Continuation-in-part of application No. 16/352,169, filed on Mar. 13, 2019.

(51) Int. Cl. A61N 5/06; A61N 1/04; A61N 1/06

(52) U.S. Cl. CPC A61N 5/0613; A61N 1/0456; A61N 1/06; A61N 2005/0643; A61N 2005/0658; A61N 2005/0652; A61N 2005/0636

(57) ABSTRACT

A system and method for a whole-body immersion bed or cabinet that permits the user to experience the therapy(s) on their entire body at the same time. The therapy in its most basic form is a combination light, and frequency therapy. Each therapy session includes a set of resonant frequencies and a set of beneficial light wavelengths applied to the user at the same time. The resonant frequencies are applied through specific pulses and/or binary codes that are chosen based upon the condition that the therapy is attempting to treat. Additionally, the resonant frequencies may be applied through a frequency pulse generator or binary code generator. The frequency pulse generator and/or binary coding generator are set to a pre-established set of frequencies that have been shown to be effective for treating each particular condition.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

Many conventional means for providing device enabled therapeutic treatment to users are designed and implemented to focus on treating the user by destroying the bug or symptom utilizing external processes and/or substances. These conventional means of device enabled therapeutic treatment to users often results in damaging the user's healthy cells in the surrounding environment of the cells that require treatment. This new damage to once healthy cells from conventional device enabled therapeutic treatment, yields newly damaged cells and side effects that create further stress on the body of the user that would not have existed but for the administration of the conventional means of device enabled therapeutic treatment to users.

Long before the dysfunction of disease or irregularity manifests physically in the body, an energetic disruption occurs. This energetic disruption might condense first in the chemistry of the body, which condenses further into the cells, which disrupts the function of the cells which cascades down into and disrupts the function of an organ which might then be recognized as disease.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of the full body treatment PBM chamber consistent with certain embodiments of the present invention.

FIG. 2 is a view of the activation and control elements of the PBM chamber consistent with certain embodiments of the present invention.

FIG. 3 is a view of the high frequency pulsing tube elements of the PBM chamber consistent with certain embodiments of the present invention.

FIG. 4 is a flow diagram for treatment operation of the system consistent with certain embodiments of the present invention.

FIG. 5 is a flow diagram for energy augmentation treatment for an individual consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Reference throughout this document to “haptic” or “haptic therapy” refers to a physical therapy that utilizes touching and pressure to apply a physical touch to an area of the human body to be treated.

Reference throughout this document to “TENS” refers to a transcutaneous electrical nerve stimulation treatment unit.

Reference throughout this document to “TENS treatment” refers to the application of electrical stimulation as a therapy treatment for an individual.

Reference throughout this document to “PEMF” refers to pulsed electromagnetic frequencies to the body that accompany photobiomodulation pulsing Reference throughout this document to “Binary Coding” refers to information coded into a specific information set of on/off sequences that might be used to seed energetic corrections.

Reference throughout this document to “Binary Code generator” refers to information coded into the photobiomodulation device that may be applied in combination with TENS or PEW′ pulsing. In a non-limiting example, a binary sequence of ‘1111000100100’ might be the code to “seed” a positive response within the body that corrects the disruption or abnormality that may precede a diagnostic of for example diabetes. A Binary Code generator is utilized to create and apply the binary coding through photobiomodulation into the body.

Modern medicine is allopathic, meaning it is focused on ‘destroying the bug’, or symptom, with an external substance or process. The challenge of this theory of treatment is that in killing the bug, the process also damages the environment and health of the cells. This new damage creates additional stress on the body and new conditions or side effects may surface. Side effects can often be as damaging as the condition being treated. Any evaluation of the use of Opioids for pain relief can attest to the dangers inherent to this allopathic process.

Modern Medicine diagnoses and augments disease from a condensed manifestation that might exhibit in pain or disease. The dense physical manifestation of pain or disease might be recognizable by a medical doctor as broken bone causing pain or measurable high blood sugar causing diabetes.

Allopathic medicine waits for the physical manifestations of pain or blood sugar to present in the patient to diagnose and then uses physical interventions of for example a cast or medication to treat the manifestation.

In an embodiment, a solution to the problems generated by allopathic methods of treatment may be found in Energy Medicine, the use of photons, electronic stimulation, binary coded information, vibration and magnetic frequencies as applied to a human body to treat conditions and speed healing. Energy method techniques utilizing the use of photons, electronic stimulation, binary coded information, vibration and or magnetic, have been shown in over 5,000 National Institutes of Health (NIH) posted studies to reduce pain, and in many cases, heal the root cause of a condition. Peer Reviewed Research is consistent in stating that these results happen free of side effects. The innovation disclosed herein presents the merger of proven technologies into a singularly applied device that creates first an energetic diagnosis, followed by a synergistic augmentation presented in the photobiomodulation chamber that amplifies the positive outcomes for a regimen of treatment; without causing any side effects as a result of the treatment. The innovation herein described, in particular embodiments, presents a novel device that merges a plurality of proven healing technologies into a single system to provide methods of diagnosis of energetic disruptions before they manifest as disease and then the device presents the proper treatment augmentation protocols based upon Energy Medicine.

The device herein described may scan and recognize energetic abnormalities in the body and then provide diagnosis specific protocols that enable a therapeutic treatment to users via the unique capacity of simultaneously administering various combinations of specialized wavelengths and intensities of light, augmented by diagnostic specific pulsing frequencies, binary coded information, and in phase electromagnetic, auditory and or TENS. The various combinations of these modalities into targetted treatment protocols is free of the side effects of modern medicine. The various combinations of energetic augmentation are designed to improve the physical condition of the user. These device enabled therpeutic treatments are designed and implemented to target individuals as a whole or individual body parts.

In an embodiment the device herein disclosed combines the ability to scan, measure and diagnose energetic disruptions in the body and then treat those energetic disruptions with the appropriate light-based frequencies delivered to a user in a full body light chamber. The light-based frequency treatment may be composed of a predetermined combination of photons using identified patterns of photobiomodulation and augmented by frequency, TENS, PEMF and or binary information patterns. Further, future scans allow the practitioner and patient to benchmark progress that might not otherwise be recognized as disease because the disease has not manifested yet physically. This early preventive detection and then alteration of energetic disruptions presents a true root cause approach to wellness.

In an embodiment, the system herein disclosed may consist of a full body light chamber with unique electronic capabilities that applies treatment to the whole body of a user enveloped within the full body light chamber. Based upon the results of a scan of the user within the full body light chamber, the system and method may diagnose issues energetically and then may treat the identified issues with appropriate frequency-based energy protocols delivered via a full body light bed. The full body light chamber, also known as a light bed, may add additional energy therapies as needed through such therapies as PEMF, TENS, and/or RadioFrequency.

Photobiomodulation (PBM) is a term that describes how light can positively stimulate cellular function through the delivery of photons of light that are absorbed into and modulate cellular function. The addition of pulsing and or resonance up to 400,000 hertz and or binary coded frequencies further direct the light to more specific diagnosis and augmentation. Research since NASA's groundbreaking work in 1996 has produced over 3,000 studies on PBM alone with over 500 Peer Reviews of the science in the intervening years. In an embodiment, the research suggests that PBM reduces cellular toxicity and inflammation while it improves energy function and circulation which in turn supports the health of the cells of a patient or user. As the cells get healthy, the benefits of good cellular health cascade down into nearly every tissue, organ and system in the body, free of negative side effects. The FDA has allowed Low Level Light Therapy (LLLT) to be exempt from regulation for minor aches and pains due to its proven non-invasive nature.

In an embodiment, Pulsed Electromagnetic Field Therapy (PEMF) likewise is supported by over 2,000 studies suggesting that pain relief, and reductions in inflammation can be triggered free of side effects.

Resonant Frequency is a term that describes how very specific frequencies may trigger an amplitude of vibration in an object. Resonant Frequencies can be used to align health within a system and or break down a pathogen. A non-limiting example of breaking down is that of a singer breaking a glass with her voice. The very specific vibration (sound) can create a resonant vibration that literally vibrates the object (glass) into breaking

Studies conducted as to the effects of Resonant Frequency are now showing that very specific resonant frequencies using PEMF can break apart pathogens. Cancer cells for example have been shown to burst when their matching resonant frequency is applied through an electromagnetic pulse. In another non-limiting example, cancer cells have been shown to have very specific resonant frequencies that range between 100,000-400,000 hertz. The device herein described has been designed to pulse photons at up to 400,000 hertz and present binary coded information utilizing PBM to ‘seed’ a positive corrective outcome.

The innovative system utilizes full body Photobiomodulation chambers, or light beds, having PEMF, Radio Frequency (RF), and TENS capabilities and adding a resonant frequency or binary code generator that modulates any combination of 5 available photonic wavelengths. The light bed may generate a full body or directed pulse delivery system composed of any of combination PBM with Electronic, Radio and/or Electromagnetic Frequencies (EMF's) therapeutic energies into the affected cells with the intent of targeting and destroying diseased cells, while at the same time creating a more healthy environment for the tissues, organs and systems of the body to thrive and multiply without interference from the body's immune system response.

The system may include, but is not limited to, a mechanism and/or device, such as, in a non-liming example, a light bed, that is in contact with the whole human body, scans the individual and reports the individual's human energy field to the individual or a therapist or other practitioner. The system may then identify any energetic disruptions or energetic pathogens as compared to baseline energy levels and energy fields established for the human body.

In an embodiment, the system herein presented is a whole body and locational, pulse and binary code directed and multi-pulse system that unites energies from waves of photons with electrical, radio and or electromagnetic waves where the energies generated are applied to the whole body of an individual in a light bed. The system first identifies any energetic disruptions by scanning the body energy patterns of the user within the light bed, as previously noted. Corrections for the identified energetic disruptions or any identified energetic pathogens may be identified and/or calculated by a software algorithm that actively manages the identification of therapeutic corrections that may be applied by the light bed. The corrections may either seed positive stimulation via binary codes or create resonance to destroy pathogens via pulsing sets. If required, the light bed may then augment the therapeutic corrections applied in the energetic field using a combination of energetic therapies which may include but are not limited to Photobiomodulation and/or TENS and/or PEW and/or Radiofrequency.

The system may use proven protocols to the dual intent of breaking down injured, damaged or diseased cells and stimulating the health of the body thus destroying energetic pathogens and resolving energy disruptions as well as physical manifestations of disease. In locational energy delivery, the system utilizes 32 control boards designed to focus different pulse rates at different organs. In a non-limiting implementation, the entire body for example might be hit with an anti-inflammatory pulse rate of 40 hertz while a stomach cancer would be targeted with a directed pulse that matches the exact resonant frequency of the cancer cell at any pulse rate necessary up to 400 KiloHertz.

In a pulse directed energy delivery, the chosen LEDs are selected for wavelengths to focus on a particular condition, such as, in a non-limiting example, a condition such as stomach cancer. To direct the focus the wavelengths would be pulsed to generate the resonant frequency of the cancer cells of the stomach cancer to break them down and shrink any cancerous mass. As part of pulse directed energy delivery, the energy may be delivered in a multi-pulse implementation. In this embodiment the system may change the pulse rate either per wavelength or per location. In a non-limiting example, stomach cancer may respond better to a 10.577 megahertz pulse with an 810 nm near infrared LED source, while a 40 hertz pulse may be delivered to the remainder of the body utilizing 528, 630, 660 and 850 nm LED sources to provide an anti-inflammatory effect.

In an embodiment, research labs have discovered evidence to show that the combination of light therapy with electromagnetic waves may create a synergy by combining the delivery of light therapy and electromagnetic waves simultaneously. The electrical and electronic components in the instant system from the outset have been designed to mimic whole body or localized pulse directed wavelengths of light to positively affect tissues, organs and systems.

In an embodiment, the innovation provides therapy to a user within the whole-body immersion device, such as a light bed, in its most basic form as a combination of light, electronic, electromagnetic and sound therapy. However, the therapy may also include haptic or rhythmic touch capability as well.

Each therapy session includes a set of resonant or binary coded seed frequencies and a set of beneficial light wavelengths applied to the user at the same time. The resonant and binary frequencies are applied through specific pulses that are chosen based upon the condition that the therapy is attempting to treat. Additionally, the resonant frequencies may be applied through pulsed electromagnetic frequency methods using a frequency pulse generator and/or a binary code generator. The frequency pulse generator is set to a pre-established set of frequencies that have been shown to be effective for treating each particular condition. The binary code generator has been set to diagnostic specific codes shown to be effective in treating a specific condition. In a non-limiting example, a plasma wave may be combined with PBM waves in phase generated at 27.15 KiloHertz to target particular conditions that respond to this frequency. The system combines therapies to generate treatment synergies. Conditions to be treated and the set of resonant frequencies to which a particular condition responds have been pre-determined and will be imported as information to be used during therapeutic sessions.

In an embodiment, at the same time that a pulsed electromagnetic frequency is applied to a patient, a set of light wavelengths is selected and the user bathed in the set of light wavelengths. The set of combined wavelengths, referred to as polychromatic multi-wave therapy, should be defined very broadly as having red, green, blue and infrared components, but not being restricted to one particular set of light wavelengths but rather selected for the treatment of a pre-determined condition.

In an embodiment, the system can measure heart rate variability when a patient is in the whole-body chamber as a means of achieving coherence when heart rate becomes steadier. Steadier is defined as the decrease in the variability of the user's heart rate as measured by the duration and variance in the active beat of the heart and the time between the active beats. A sensor that monitors and captures the user's heart-beat, heart rate, and time between beats may be incorporated into the system to provide data for the analysis of a user's heart beat to determine when the user's heart rate becomes steadier as a function of the therapeutic light wavelengths being applied to the user when the user is inside the whole-body chamber and the therapy is active. In a non-limiting example, this sensor may be configured as a portion of an intranasal connective element that is attached to one or both nostrils of the user's nose and is in data communication, either wired or wirelessly, with the whole-body chamber. In a non-limiting example, the intranasal connective element is active to send duplicate frequencies and photons into the blood that travels to and feeds the brain of the user when inside the whole-body chamber.

In an alternative embodiment, the sensor that is active to monitor and capture the user's heart-beat, heart rate, and time between beats may be configured as an electrode that may be placed onto the ear lobe. This electrode attached to the earlobe may be used to measure heart rate and coherence of the measured heart rate parameters while the user is in the full-body chamber.

Additionally, a pulsed wavelength at 528 Hertz may be applied to form the haptic portion of the beneficial therapy in combination with the light and PMF therapies. The 528 Hertz frequency may be generated by speakers external to the container in which the patient is situated. The 528 Hertz frequency is the frequency at which water will vibrate, thus transmitting the pulse into human tissues as a touch. During the creation of a treatment plan for a particular condition, the plan may include the use of an electrode affixed to the user's earlobe to collect and record physiological data during treatment and the use of double pulses of light during treatment as a specific technique to which some conditions may be responsive.

### System

The device herein disclosed is a full body LED Light Chamber having the dimensions of approximately 86.5″ long, approximately 37″ wide, and approximately 38″ high. The whole-body treatment device may have an Aluminum Alloy Frame with 10993 biocompatible acrylic. The treatment device may also comprise the following physical elements of:
220 Volt 30 Amp power supply;
Onboard computer having a processor and memory;
Pulse Generator loaded with diagnostic and augmentation protocols;
Electronic Scanning device to measure and benchmark energetic condition;
Binary Code Generator loaded with diagnostic specific protocols to augment Scanning;
32 Individual PCBs to direct the wavelength, pulse and location;
2 Power Bus boards;
2 Signal Bus boards;
12-volt power supplied for controls and computers;
5000 watts/48 V transformer powering 2304 LED's divided into 6 strings of lights producing the following wavelengths of light:
   1×528 nm Green
   1×633 Red
   1×660 Red
   2×810 NIR
   1×850 NIR

In a non-limiting example, the system may also comprise a conductive plate as the physical element of the system that is placed in contact with the body of the individual undergoing examination.

### Treatment:

In an embodiment, non-medical professionals would be offered a treatment device with 7-15 pulse directed protocols that do not require radiant frequency matching. These frequencies are applicable to the entire body free of negative side effects. Treatment protocols established for the device would include but not be limited to protocols for treating inflammation, stress relief, sleep, energy, mood, pain and grounding.

In an embodiment, medical professionals would be offered the pulse wave generator treatment device having greater capability and capacity for the treatment of various conditions. This treatment device could be configured with up to 2000 tested frequency protocols, each containing from 30-300 frequencies to be applied during a 15-90 minute treatment time, the number and wavelength of selected frequencies pre-configured for the condition to be treated during the treatment session. In these treatment methods, resonant frequencies target disease while PBM triggers cellular health.

In a non-limiting example, the pulse wave generator may be configured to deliver a particular pulse rate to the light bed to treat a particular condition. In this example a pulse wave generator may be configured to deliver a pulse rate of 89.53 hertz to create a resonance that breaks down a viral pathogen. Other conditions may be treated by alternative pulse rates based upon the type of condition or pathogen to be targeted.

In an embodiment, the system may also contain a plasma wave generator that pulses matching electromagnetic frequencies into the room in Double Pulse Resonant Frequency (DPRF) mode. This mode of operation may provide the ability to more deeply and consistently deliver the therapeutic healing frequencies into the tissues of the user. The plasma wave generator enables DPRF mode of therapy.

In an embodiment, the room housing the PBM chamber and PEMF generator would be built as a Faraday cage insulating the energies released in the room from the external environment. In this fashion external energies may not penetrate the treatment room to interfere with the therapeutic energies released during a user's immersion in the PBM chamber. The Faraday cage housing surrounding the treatment space also serves to contain the treatment energies within the treatment room for the benefit of the user of the system, insulating against outside interference and encapsulating the beneficial energies within the treatment space for the benefit of the user.

In an embodiment, the diagnostic treatment may be delivered as a result of energetic disruptions detected by a scan of the individual undergoing examination through the use of a conductive plate, or other scanning device. A software-based analytic algorithm is utilized to compare and calculate energetic differences from established human normal levels. If energetic disruptions or pathogens are detected during the scan the system may utilize the collected data from the diagnostic scan as a comparison against normative energy levels for a human to calculate and program energetic corrections into the energetic augmentation device which augments through the application of any combination of photobiomodulation, and/or TENS, and/or PEMF, and/or radiofrequency treatments.

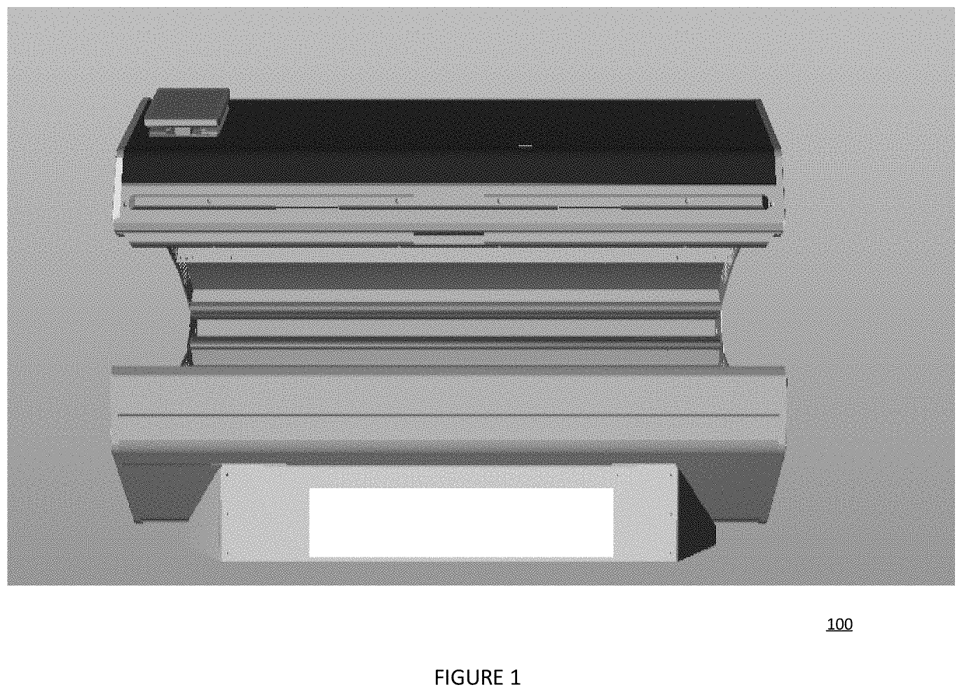

Turning now to FIG. 1, this figure presents a view of the full body treatment PBM chamber consistent with certain embodiments of the present invention. In an exemplary embodiment, the full body chamber 100 is configured to surround a user when involved in therapeutic action. The top portion of the full body chamber 100 is attached to one or more hinges (not shown) to permit the top portion to swing up and out of the way as a user enters the full body chamber 100, lying prone on the bottom portion of the chamber. Prior to the inception of the therapy session, the top portion is pulled down toward the user lying in the full body chamber such that the top side and the bottom side of the user will receive the benefit of the therapeutic healing frequencies during the therapy session.

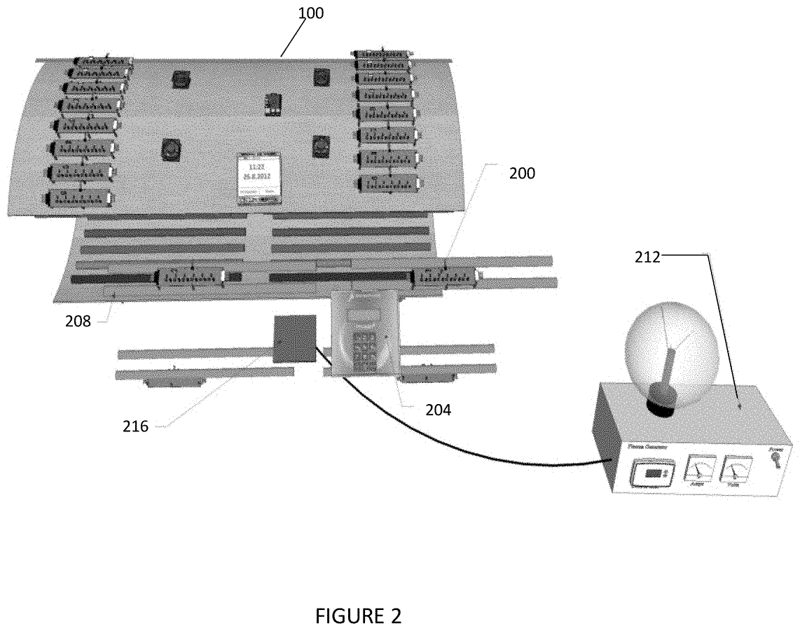

Turning now to FIG. 2, this figure presents a view of the activation and control elements of the PBM chamber consistent with certain embodiments of the present invention. In an exemplary embodiment, the system presents the physical elements that provide the treatment through photobiomodulation and resonant or binary frequencies. In an embodiment, the full body chamber may have 32 pulsing control boards 200 to focus different pulse rates at different organs, as well as providing an overall pulse to bathe the entire body in a single frequency. The pulsing control boards 200 may be under control of a medical practitioner or a non-medical practitioner user through the pulsing control pad 204. The pulsing control pad 204 may provide the user controls to set an automated program in place or permit a user to input sets of desired frequencies based upon the result the user desires to achieve. In a non-limiting example, a user may select a whole-body pulse at a 40 Hertz frequency while simultaneously targeting a disease or organ with a directed pulse that matches the resonant frequency of the targeted organ or targeted disease cells.

In an embodiment, the pulsing control pad 204 may direct the high frequency pulsing tubes 208 to select the frequencies to be delivered from each of the high frequency pulsing tubes 208 during a therapy session. Simultaneously, a plasma generator 212 may be active to deliver pulses of matching electromagnetic frequencies into the room in Double Pulse Resonant Frequency (DPRF) mode. The plasma generator 212 is attached to the full body chamber 100 through an interface board 216 to permit the synchronization between the pulsed frequency treatment and the DPRF treatment mode. The combination of treatments provides both light and sound frequencies targeted to conditions associated with the user contained within the full body chamber 100.

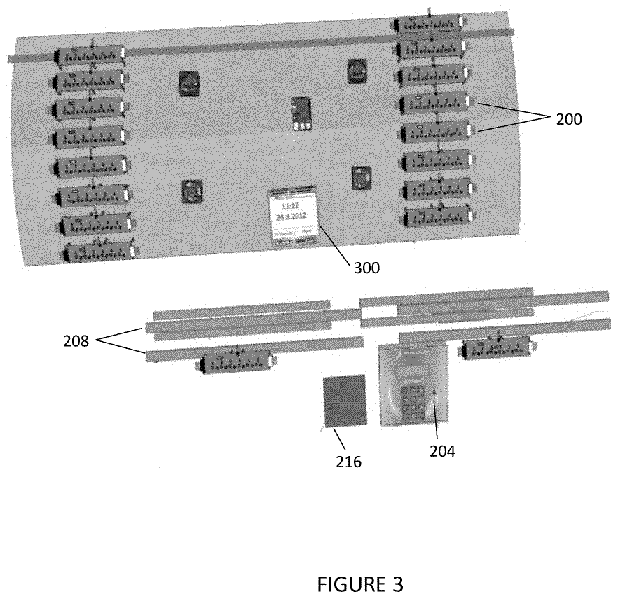

Turning now to FIG. 3, this figure presents a view of the high frequency pulsing tube elements of the PBM chamber consistent with certain embodiments of the present invention. In an exemplary embodiment, the pulse control elements of the full body chamber are presented in isolation. In this embodiment, the high frequency pulsing control boards 200, high frequency pulsing tubes 208, pulsing control 204, and the board interface 216 to permit synchronization with a plasma generator are presented as a separate system for delivery of light therapy through specific light frequencies. Additionally, a user interface display 300 permits informational displays to a user that may include timing, frequency selections, or other informational displays to assist a user during the therapy experience.

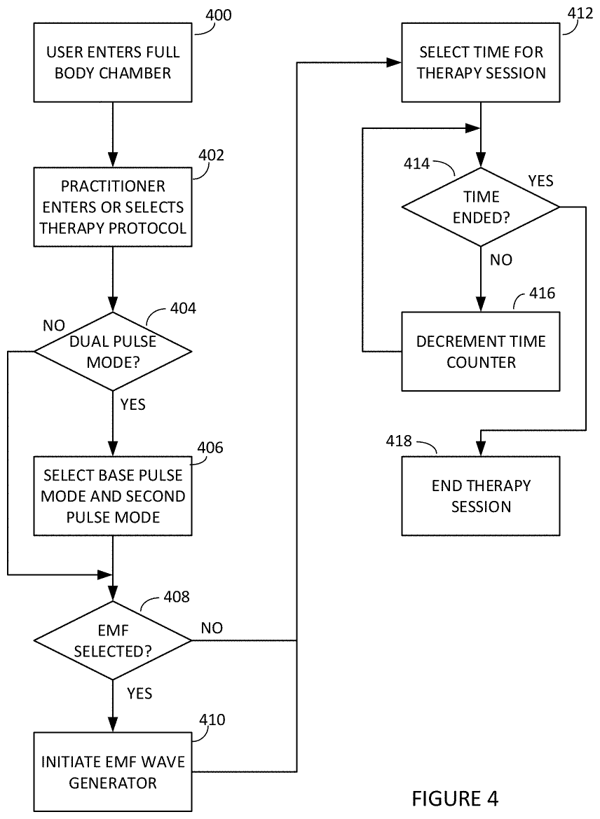

Turning now to FIG. 4, this figure presents an operational flow diagram for treatment operation of the system consistent with certain embodiments of the present invention. In this embodiment, at 400 the treatment for a user would begin with a user entering the full body chamber and reclining in the treatment chamber in such a way as to permit the treatment therapies to encompass the entirety of the user's body. At 402, a practitioner, or alternatively the user, serving as their own practitioner, may select a pre-stored protocol for a particular treatment, or may enter parameters for a particular desired therapy session. At 404, the practitioner or user may select either single pulse mode or dual pulse mode for the given therapy session. If the practitioner or user selects dual pulse mode delivery for the given therapy session at 406 the practitioner or user may select a base pulse mode for the entirety of the body such as, in a non-limiting example, an anti-inflammatory pulse rate of 40 hertz, and select a different poly-wavelength therapy to target a particular condition. If the practitioner or user selects a single pulse mode, the practitioner or user may instead enter either a single base pulse mode or a poly-wavelength mode for delivery of the therapeutic wavelengths of light and sound.

At 408, the practitioner or user may select the addition of EMF therapy to the dual or single pulse mode selected for the therapy session. If the practitioner desires to add EMF therapy to the session, at 410 an EMF wave generator will be engaged and synchronized with the previously selected wavelength therapy to deliver both light and electromagnetic pulses to the user during the therapy session. If the practitioner or user does not desire to add EMF therapy to the session the EMF wave generator will remain unengaged during the therapy session. At 412, the practitioner or user may be prompted to enter a time limit for the duration of the therapy session. The practitioner or user may enter the time limit and initiate the therapy session requested.

At 414, the system may check to determine whether the therapy session time limit has expired. At 416, if the therapy session time limit has not expired, the system will decrement the timer and continue the therapy session, checking the time limit periodically. At 418, if the therapy session time has elapsed the system will end the therapy session by ending the light poly-wavelength and/or emf delivery to the user through the full body chamber.

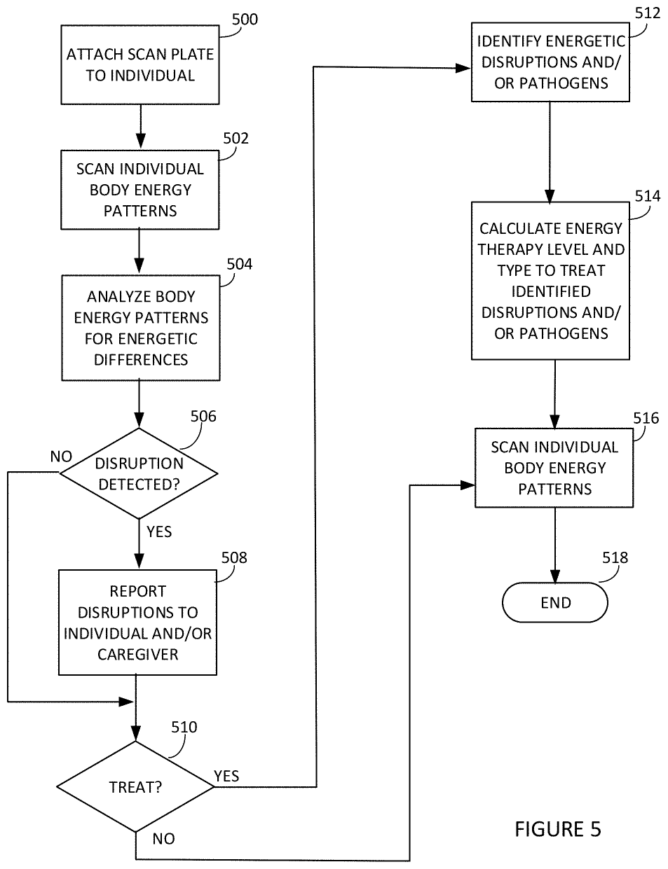

Turning now to FIG. 5, this figure presents a flow diagram for energy augmentation treatment for an individual consistent with certain embodiments of the present invention. In this embodiment, at 500 to begin the examination of an individual a sensor device such as, in a non-limiting example, a scan plate having the ability to perform an active energy scan is attached to the individual through straps or other removable means. At 502 the sensor device performs a scan of the body energy patterns for the individual undergoing examination. At 504 the system initiates an algorithmic comparison to analyze the body energy patterns of the individual against normative values for a human body to identify energetic difference such as energy disruptions and/or energy pathogens. At 506, if the algorithm as engaged detects and identifies such energy disruptions and/or energy pathogens, the algorithm may then report the identified energy disruption and/or energy pathogen findings to the individual, a caregiver, a therapist, or other qualified party at 508. Regardless of whether energy disruptions and/or pathogens are detected by the algorithm, the individual is provided with the option to request treatment. The system at 510 provides the individual, or caregiver, therapist or other qualified party, the option to request treatment. The individual may request or deny treatment at 510 based upon their desire to receive such treatment at that time.

If an individual requests treatment at 512 the system will identify the energetic disruptions and/or pathogens and classify them to determine what treatment is required to correct the energetic disruption and/or to eliminate the determined energy pathogens. At 514 the system may calculate the energy therapy levels and treatment types to resolve the issues identified. The system may choose from treatments delivered as photobiomodulation, TENS, PEW, radiofrequency treatments, pulsed waves, or any combination of these treatments as required to resolve the issues of energetic disruption and/or energetic pathogens as identified and classified by the system. At 516, the system may perform another scan of the individual after delivery of the selected and calculated therapeutic effects to determine the efficacy of the delivered treatment and to determine if additional therapies may be required to completely treat the energy disruptions and/or energetic pathogens. The additional scan may be reported to the individual, caregiver, therapist, or other authorized party as feedback for future therapy considerations.

If the individual at 510 chose not to receive treatment or if the treatment is concluded the system then ceases operation at 518.

While certain illustrative embodiments have been described, it is evident that many alternatives, modifications, permutations and variations will become apparent to those skilled in the art in light of the foregoing description.

We claim:

1. A system for therapeutic treatment, comprising:
a light therapy chamber suitable to provide treatment to the entire body of a user;
a sensor that is attached to said user;
said sensor collecting information from said user by scanning for energetic disruptions;
an algorithm receiving said collected information from said sensor;
said algorithm identifying energetic disruption levels and calculating energy therapy levels required to treat said energetic disruptions;
said algorithm directing said light therapy chamber to apply calculated energy therapy levels to said user through application of any of predetermined patterns of photobiomodulation augmented by frequency and/or binary information patterns;
where the energy therapy level treatments are designed to treat a whole body and/or a specific body portion of a user simultaneously.

2. The system of claim 1, where the application of calculated energy therapy levels is performed through a combination of any of photobiomodulation, TENS, PEMF and/or radiofrequency treatments.

3. The system of claim 1, where the photobiomodulation therapy comprises generating bio-resonant whole-body frequency and pulsed frequencies forming a combination therapy.

4. The system of claim 1, further comprising an electrode connected to the user within the light therapy chamber to monitor and collect heart rate, heart-beat, and time between heart beats data of the user.

5. The system of claim 1, where said sensor comprises at least a conductive plate.

6. The system of claim 1, further comprising said scan further scans for energetic pathogens.

7. The system of claim 5, where said scan is analyzed to identify said energetic pathogens and an energy therapy level is calculated to treat said identified energetic pathogens.

8. The system of claim 2, where said any of photobiomodulation, TENS, PEMF and/or radiofrequency treatments are selected to target a particular body portion, set of cells, or user condition to deliver the desired treatment effect.

9. The system of claim 1, where the pulse patterns are created and delivered by a plasma wave generator associated with the light therapy chamber.

10. The system of claim 1, where the sets of frequencies selected for delivery to the user comprise any or all of five light wavelengths designed to provide a particular therapeutic effect on the user.

11. A method for therapeutic treatment, comprising:
a sensor that is attached to a user collecting information from said user by scanning for energetic disruptions;
an algorithm receiving said collected information from said sensor;
said algorithm identifying energetic disruption levels and calculating energy therapy levels required to treat said energetic disruptions;
said algorithm directing said light therapy chamber to apply calculated energy therapy levels to said user through application of any of predetermined patterns of photobiomodulation augmented by frequency and/or binary information patterns;
where the energy therapy level treatments are designed to treat a whole body and/or a specific body portion of a user simultaneously.

12. The method of claim 11, where the application of calculated energy therapy levels is performed through a combination of any of photobiomodulation, TENS, PEMF and/or radiofrequency treatments.

13. The method of claim 11, where the photobiomodulation therapy comprises generating bio-resonant whole-body frequency and pulsed frequencies forming a combination therapy.

14. The method of claim 11, further comprising an electrode connected to the user within the light therapy chamber to monitor and collect heart rate, heart-beat, and time between heart beats data of the user.

15. The method of claim 11, where said sensor comprises at least a conductive plate.

16. The method of claim 11, further comprising said scan further scans for energetic pathogens.

17. The method of claim 15, where said scan is analyzed to identify said energetic pathogens and an energy therapy level is calculated to treat said identified energetic pathogens.

18. The method of claim 12, where said any of photobiomodulation, TENS, PEMF and/or radiofrequency treatments are selected to target a particular body portion, set of cells, or user condition to deliver the desired treatment effect.

19. The method of claim 11, where the pulse patterns are created and delivered by a plasma wave generator associated with the light therapy chamber.

20. The method of claim 11, where the sets of frequencies selected for delivery to the user comprise any or all of five light wavelengths designed to provide a particular therapeutic effect on the user.
